(19) United States
(12) Patent Application Publication
(10) Pub. No.: US 2010/0298683 A1
(43) Pub. Date: Nov. 25, 2010

(54) PHYSIOLOGICAL MONITORING DEVICES AND METHODS

(75) Inventors: Ronald J. CABRERA, Mission Viejo, CA (US); James W. Hill, Mission Viejo, CA (US)

Correspondence Address: CROCKETT & CROCKETT, P.C., 26020 ACERO, SUITE 200, MISSION VIEJO, CA, 92691, US

(73) Assignee: Deltin Corporation, A California Corporation, Mission Viejo, CA (US)

(21) Appl. No.: 12/212,545

(22) Filed: Sep. 17, 2008

(51) Int. Cl. A61B 5/145; G08B 23/00

(52) U.S. Cl. 600/364; 340/573.1

(57) ABSTRACT

Devices and methods are described for wirelessly monitoring an emergency responder. In some embodiments, a sensor acquires values of carboxyhemoglobin in blood. The values are recorded and are used to provide feedback to a user. The feedback includes at least one of visible, tactile, and audible information.

### FIELD OF THE INVENTION

Embodiments of the inventions generally relate to physiological monitoring devices and methods and, in particular, relate to devices and methods for wirelessly monitoring an emergency responder.

### BACKGROUND OF THE INVENTION

Emergency personnel face many hazards, including exposure to carbon monoxide. Because carbon monoxide is colorless, odorless, and attaches to hemoglobin at a rate approximately 200 times greater than does oxygen, in the presence of carbon monoxide the human body may be deprived of oxygen. Effects of exposure may include dizziness, increased heart rate, confusion, even death, and may have detrimental consequences for cognition and mental processes for months or years later, or even permanently. Fire fighters are especially susceptible to the effects of carbon monoxide due to their work environment.

While working in places with limited ventilation, such as building fires, fire fighters typically wear self-contained breathing apparatuses (SCBA units) that provide breathing oxygen through compressed air delivery. In the case of brush fires, however, emergency responders often do not wear SCBA units due to the large geographical areas involved, the limited mobility that forced air systems impose on users, and because it is impractical to exchange compressed air cylinders across wide geographies and for recurring time periods. Fire fighting is strenuous activity, and when required to wear an SCBA unit, a typical user's air cylinder(s) are depleted in much less than an hour.

Even after exposure and inhalation of certain levels of smoke and other air pollutants, individual fire fighters are often remiss to remove themselves from fighting a fire. Supervisors are often at the mercy of individual fire fighter's self-reporting on the symptoms of carbon monoxide exposure, and even individual fire fighters are typically at the mercy of how he or she may ‘feel’ after exposure to air borne pollutants.

To some degree, a healthy fire fighter may safely face exposure to levels of carbon monoxide that are known to not be life threatening while staying on the fire fighting line to continue containing or extinguishing the fire. However, shifting winds, exposure to consistent levels of carbon monoxide, physical exertion, loss of body fluids, and many other factors may combine or reach a point where it is no longer safe or wise for an otherwise healthy fire fighter to stay on the fire fighting line.

### SUMMARY OF THE INVENTION

In accordance with certain embodiments, devices and methods for monitoring an emergency responder are provided. In certain embodiments the devices and methods may apply to providing a user with feedback on any carboxyhemoglobin detected in at least one emergency responder. In certain embodiments the user may be the person being monitored, and in certain embodiments, the user may be located remotely from the person being monitored. In certain embodiments the devices and methods may apply providing feedback on a value comprising carboxyhemoglobin levels concurrent to another value selected from the group consisting of heart rate, a peripheral perfusion index, and environmental carbon monoxide levels, and providing feedback the values to a user (whether the user is a remotely located supervisor or the person being monitored). Certain embodiments may comprise a transmitter coupled to a helmet, and a remote receiver. In certain embodiments a receiver is configured to receive wireless signals comprising values of at least one of detected levels of carboxyhemoglobin, heart rate, a peripheral perfusion index, and/or environmental carbon monoxide levels. The feedback is provided so that the user may fully and safely manage available human resources (e.g., safely manage the fire fighters on the fire fighting line).

In a certain embodiment, a method is provided for monitoring emergency personnel. The method includes acquiring values of levels of carboxyhemoglobin in a person's blood during a sampling period, the person being in a region near or at a source of carbon monoxide. The method also includes recording the values on machine-readable media, determining a regression function for the values with respect to time, determining at least one of an attribute of the function and a future value of a carboxyhemoglobin level in the person's blood, the future value being determined by extrapolation of the function, and providing a user with safety information based on the at least one of the attribute of the function and the future value. In the method, the safety information includes at least one of visible, tactile, and audible information. The safety information includes at least one of a time of remaining safety in the region, a warning to exit the region, an indication of continuing safety in the region, and an indication of impending danger. In a certain embodiment, the attribute of the function includes at least one of a derivative and an integral. In a certain embodiment, the user is the person being monitored. In a certain embodiment, a machine-readable medium has machine-executable instructions for performing the method.

In a certain embodiment, a method is provides for monitoring emergency personnel. The method includes, during a sampling period and of a person in a region near or at a source of carbon monoxide, acquiring (a) values of levels of carboxyhemoglobin in the person's blood, and (b) a value of a parameter selected from the group consisting of the person's heart rate, an indicator of the person's peripheral perfusion, and ambient carbon monoxide. The method includes recording the values of (a) and (b) on machine-readable media, determining a regression function for at least one of: (i) each of (a) and (b) with respect to time; and (ii) a combination of (a) and (b) with respect to time; determining at least one of: (i) an attribute of the function; and (ii) a future value of at least one of (a) and (b), the future value being determined by extrapolation of the function, and providing a user with safety information based on the at least one of the attribute of the function and the future value of at least one of (a) and (b). The safety information includes at least one of visible, tactile, and audible information. The safety information also includes at least one of a time of remaining safety in the region, a warning to exit the region, an indication of continuing safety in the region, and an indication of impending danger. In certain embodiments, the attribute of the function includes at least one of a derivative and an integral. In certain embodiments, the user is the person being monitored. In certain embodiments, the method includes determining expected levels of carboxyhemoglobin present in the person based on the value of ambient carbon monoxide. In certain embodiments, determining expected levels of carboxyhemoglobin includes processing the environmental carbon monoxide levels such that % expected COHb=(3.317×10⁻⁵) (ppm CO)^{1.036 (RMV) (t)}, where: ppm CO=environmental carbon monoxide levels in parts per million; RMV=an expected respiratory minute volume of air breathed by the at least one emergency responder in liters per minute; and (t)=exposure time for the at least one emergency responder in minutes. Certain embodiments include a machine-readable medium containing machine-executable instructions for performing the method.

In a certain embodiment, a device for monitoring emergency personnel is provided. The device includes means for acquiring values of levels of carboxyhemoglobin in a person's blood during a sampling period, the person being in a region near or at a source of carbon monoxide. The device also includes means for recording the values on machine-readable media, means for determining a regression function for the values with respect to time, means for determining at least one of an attribute of the function and a future value of a carboxyhemoglobin level in the person's blood, the future value being determined by extrapolation of the function, and means for providing a user with safety information based on the at least one of the attribute of the function and the future value. The safety information includes at least one of visible, tactile, and audible information. The safety information also includes at least one of a time of remaining safety in the region, a warning to exit the region, an indication of continuing safety in the region, and an indication of impending danger. In certain embodiments, the attribute of the function includes at least one of a derivative and an integral. In certain embodiments, the user is the person being monitored. Certain embodiments include a machine-readable medium containing machine-executable instructions for performing steps for each of the means in the device.

In a certain embodiment, a device for monitoring emergency personnel is provided. The device includes means for acquiring, during a sampling period and for a person in a region near or at a source of carbon monoxide, (a) values of levels of carboxyhemoglobin in the person's blood, and (b) a value of a parameter selected from the group consisting of the person's heart rate, an indicator of the person's peripheral perfusion, and ambient carbon monoxide. The device also includes means for recording the values of (a) and (b) on machine-readable media, means for determining a regression function for at least one of: (i) each of (a) and (b) with respect to time; and (ii) a combination of (a) and (b) with respect to time; means for determining at least one of: (i) an attribute of the function; and (ii) a future value of at least one of (a) and (b), the future value being determined by extrapolation of the function; and means for providing a user with safety information based on the at least one of the attribute of the function and the future value of at least one of (a) and (b). The safety information includes at least one of visible, tactile, and audible information. The safety information also includes at least one of a time of remaining safety in the region, a warning to exit the region, an indication of continuing safety in the region, and an indication of impending danger. In certain embodiments, the attribute of the function includes at least one of a derivative and an integral. In certain embodiments, the user is the person being monitored. In certain embodiments, the device includes determining expected levels of carboxyhemoglobin present in the at least one emergency responder based on ambient carbon monoxide. In certain embodiments, determining expected levels of carboxyhemoglobin comprises processing the ambient carbon monoxide levels such that % expected COHb=(3.317×10⁻⁵) (ppm CO)^{1.036 (RMV) (t)}, where: ppm CO=environmental carbon monoxide levels in parts per million; RMV=an expected respiratory minute volume of air breathed by the at least one emergency responder in liters per minute; and (t)=exposure time for the at least one emergency responder in minutes. Certain embodiments include a machine-readable medium containing machine-executable instructions for performing steps for each of the means in the device.

In a certain embodiment, a device for monitoring emergency personnel is provided. The device includes a sensor configured to acquire values indicative of levels of carboxyhemoglobin in a person's blood during a sampling period, the person being in a region near or at a source of carbon monoxide. The device also includes a processor configured to determine a regression function for the values with respect to time, and to determine at least one of an attribute of the function and a future value of a carboxyhemoglobin level in the person's blood, the future value being determined by extrapolation of the function. The device also includes an output device configured to provide a user with safety information based on at least one of the attribute of the function and the future value; wherein the safety information comprises at least one of visible, tactile, and audible information; and further wherein the safety information comprises at least one of a time of remaining safety in the region, a warning to exit the region, an indication of continuing safety in the region, and an indication of impending danger. In certain embodiments, the attribute of the function comprises at least one of a derivative and an integral. In certain embodiments, the user is the person. In certain embodiments, the device also includes a receiver coupled with the processor; and a transmitter, wherein the transmitter transmits the acquired values to the receiver, and the receiver conveys the acquired values to the processor as inputs for at least the regression function. In certain embodiments, the device also includes a receiver coupled with the processor; and a transmitter, wherein the transmitter transmits at least one of the attribute of the function and the future value to the receiver, and the receiver conveys the at least one of the attribute of the function and the future value to the processor as inputs for the safety information. In certain embodiments, the device also includes a receiver coupled with the processor and the output device; and a transmitter, wherein the transmitter transmits the safety information to at least one of the receiver and the output device, and, if the receiver receives the safety information, the receiver conveys the safety information to at least one of the processor and the output device.

In a certain embodiment, a device for monitoring emergency personnel is provided. The device includes a sensor configured to acquire during a sampling period and of a person in a region near or at a source of carbon monoxide, (a) values of levels of carboxyhemoglobin in the person's blood, and (b) a value of a parameter selected from the group consisting of the person's heart rate, an indicator of the person's peripheral perfusion, and ambient carbon monoxide. The device also includes a processor configured to determine a regression function for at least one of: (i) each of (a) and (b) with respect to time; and (ii) a combination of (a) and (b) with respect to time. The processor is also configured to determine at least one of: (i) an attribute of the function; and (ii) a future value of at least one of (a) and (b), the future value being determined by extrapolation of the function; and an output device configured to provide a user with safety information based on the at least one of the attribute of the function and the future value; wherein the safety information comprises at least one of visible, tactile, and audible information; and further wherein the safety information comprises at least one of a time of remaining safety in the region, a warning to exit the region, an indication of continuing safety in the region, and an indication of impending danger. In certain embodiments, the attribute of the function comprises at least one of a derivative and an integral. In certain embodiments, the user is the person. In certain embodiments, the processor is further configured to determine expected levels of carboxyhemoglobin present in the person based on the value of ambient carbon monoxide. In certain embodiments, the device further includes a receiver coupled with the processor; and a transmitter, wherein the transmitter transmits the acquired values to the receiver, and the receiver conveys the acquired values to the processor as inputs for at least the regression function. In certain embodiments, the device further includes a receiver coupled with the processor; and a transmitter, wherein the transmitter transmits at least one of the attribute of the function and the future value to the receiver, and the receiver conveys the at least one of the attribute of the function and the future value to the processor as inputs for the safety information. In certain embodiments, the device also includes a receiver coupled with the processor and the output device; and a transmitter, wherein the transmitter transmits the safety information to at least one of the receiver and the output device, and, if the receiver receives the safety information, the receiver conveys the safety information to at least one of the processor and the output device. In certain embodiments determining expected levels of carboxyhemoglobin comprises processing the environmental carbon monoxide levels such that % expected COHb=(3.317×10⁻⁵) (ppm CO)^{1.036 (RMV) (t)}, where: ppm CO=environmental carbon monoxide levels in parts per million; RMV=an expected respiratory minute volume of air breathed by the at least one emergency responder in liters per minute; and (t)=exposure time for the at least one emergency responder in minutes.

### BRIEF DESCRIPTION OF THE DRAWINGS

The inventions, both to their organization and manner of operation, may be further understood by reference to the drawings that include FIGS. 1 through 8 taken in connection with the following descriptions:

FIG. 1 is an illustration of certain embodiments comprising a transmitter coupled to a fire fighter's helmet;

FIG. 2 is an illustration of certain embodiments comprising a sensor or sensors coupled to an adjustable head band within an emergency responder's helmet (e.g., the helmet shown in FIG. 1);

FIG. 3 is an illustration of certain embodiments comprising a plurality of transmitters coupled to respective fire fighter helmets, and a personal digital assistant (PDA), where the transmitters and PDA are communicatively coupled to a transceiver;

FIG. 4 is a block diagram of certain embodiments including a transmitter, a receiver, and a PDA that are communicatively coupled to each other;

FIG. 5 is a block diagram of certain embodiments including a carboxyhemoglobin reporting system;

FIG. 6 is a chart that illustrates detected levels of carboxyhemoglobin and environmental carbon monoxide, and a linear trend of the illustrated data;

FIG. 7 is a chart that illustrates detected levels of carboxyhemoglobin and environmental carbon monoxide, and a non-linear trend of the illustrated data; and

FIG. 8 is a flowchart that illustrates a method and/or processor-executable instruction steps for various embodiments.

### DETAILED DESCRIPTION OF THE INVENTION

The following description of illustrative non-limiting embodiments discloses specific configurations and components. However, the embodiments are merely examples of the present inventions, and thus, the specific features described below are merely used to describe such embodiments to provide an overall understanding of the inventions. One skilled in the art readily recognizes that the present inventions are not limited to the specific embodiments described below. Furthermore, certain descriptions of various configurations and components of the present inventions that are known to one skilled in the art are omitted for the sake of clarity and brevity. Further, while the term “embodiment” may be used to describe certain aspects of the inventions, the term “embodiment” should not be construed to mean that those aspects discussed apply merely to that embodiment, but that all aspects or some aspects of the disclosed inventions may apply to all embodiments, or some embodiments.

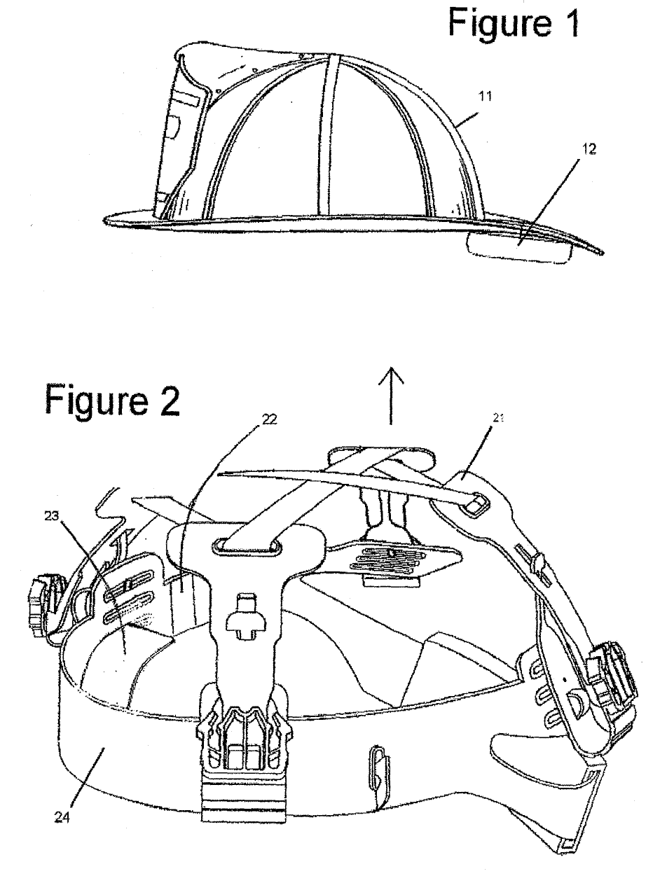

FIG. 1 is an illustration of certain embodiments comprising a transmitter 12 coupled to the rear underside portion of personnel equipment 11. Transmitter 12 may be coupled to the personnel equipment 11 using various means including a slide and lock bracket, a clip, a strap, adhesives, or other means that would be known to one of skill in the art. Transmitter 12 may be configured to operate using various wireless transmission methods, including Code Division Multiple Access, Time Division Multiple Access, or Frequency Division Multiple Access, to thereby permit multiple transmitters 12 to concurrently transmit data to one or more receivers. In certain embodiments, transmitter 12 is configured to transmit spread spectrum signals, such as Orthogonal Frequency Division Multiplexed signals.

While personnel equipment 11 is illustrated as a fire fighter's helmet in FIG. 1, other personnel equipment is envisioned as within the scope of certain embodiments. For example, in certain embodiments personnel equipment 11 may comprise military, police, or other headgear. In certain embodiments personnel equipment 11 may comprise a baseball cap, a night watchman's cap, a sweatband, a bracelet, an armband, a wristband, a glove, underclothing, over-clothing, socks, boots, pants, a shirt, a jacket, protective eye wear, or other personnel equipment that might be regularly worn or used by persons responding to an emergency situation. For example, a helmet, gloves, and/or protective eyewear may comprise personnel equipment 11 that a fire fighter may use when fighting a brush fire, among other items, and the subject technology of certain embodiments may comprise the transmitter 12 being coupled to at least one of a fire fighter's helmet, gloves, and/or protective eyewear.

FIG. 2 illustrates certain embodiments comprising a sensor 22 (or sensors 22/23) coupled to an adjustable head band 24. Adjustable head band 24 comprises a suspension assembly 21 as part of an emergency responder's helmet (e.g., the helmet shown in FIG. 1). While two sensors (22, 23) are shown in FIG. 2, in certain embodiments any number of sensors may be included, for instance, from one sensor to a half dozen or more. In certain embodiments, sensor 22 comprises a pulse oximeter sensor, for example, a LNOP TF-1 or LNCS TF-1 (or similar) reusable sensor manufactured by Masimo, Inc., of Irvine, Calif. In certain embodiments, sensor 22 comprises a disposable pulse oximeter sensor. In certain embodiments, sensor 22 comprises a pulse oximeter sensor that dangles from a connection cable (not shown) coupled to suspension assembly 21 approximately four to six inches below the adjustable head band 24 for clipping to an earlobe. For instance, the Masimo corporation LNCS TC-1 (or similar) adult ear sensor may be used in this fashion.

Pulse oximeter sensors operate based upon the red and infrared light absorption characteristics of oxygenated and deoxygenated hemoglobin in the human blood stream. Oxygenated hemoglobin absorbs more infrared light and allows more red light to pass. Deoxygenated (or reduced oxygen) hemoglobin absorbs more red light and allows more infrared light to pass. Red light has a wavelength of approximately 600-750 nm, and infrared light has a wavelength of approximately 850-1000 nm.

In certain embodiments, sensor 22 is a reflectance-type pulse oximeter sensor. For example, when sensor 22 is a forehead sensor and/or is coupled substantially directly to head band 24, a reflectance-type sensor may be employed. In these embodiments, a light emitter and a photo detector are located proximate to each other in the sensor at the measurement location, e.g., at the forehead. The red and infrared light are produced by the emitter(s), transmitted to the forehead where the red and infrared light interact with either or both of oxygenated and deoxygenated hemoglobin, and then are reflected to the photo detector(s) to obtain a reading.

In certain embodiments, sensor 22 is a transmission-type of pulse oximeter sensor, e.g., when sensor 22 is clipped to an earlobe. In these embodiments, the emitter produces red and infrared light that are transmitted through the earlobe to a photo detector on the opposite side of the ear lobe. The transmitted red and infrared light interact with either or both of oxygenated and deoxygenated hemoglobin, and are then received by the photo detector to obtain a reading.

After the transmitted red and infrared signals pass through the tested site and are received at the photo detector, a ratio of received red-to-infrared light is calculated. This ratio may then be compared to a look-up table that enables conversion of the ratio to an accurate saturation of oxygen in arterial blood flow, or an SpO2 value (also called an SaO2 value). In certain embodiments, a red to infrared ratio of 0.5 equates to approximately 100% SpO2, a ratio of 1.0 equates to approximately 82% SpO2, and a ratio of 2.0 equates to 0% SpO. The red to infrared ratio may also relate to a level of carboxyhemoglobin (COHb, or hemoglobin that has combined with carbon monoxide) within the blood stream.

All pulse oximetry measuring sites include light absorbers such as skin, tissue, venous blood, and arterial blood. Skin, tissue, and venous blood are fairly constant light absorbers in that they absorb substantially the same amount of light regardless of changes in the amount of blood flow. In contrast, arterial blood has a substantial fluctuation of light absorbed during changes in amounts of blood flow, for example, between each time the heart beats and each time the heart rests between beats. When the heart beats, it contracts causing a surge of arterial blood. The surge of arterial blood momentarily (and perhaps not immediately) increases arterial blood volume across the measuring site. The increase in arterial blood volume results in greater light absorption during the surge. Between heart beats, there is a lessening in the arterial blood flow. In certain embodiments sensor 22 subtracts the detected values of lessened light absorption between heart beat periods from the detected values of greater light absorption during heart beat periods, to thereby derive a midpoint between a peak and a trough, and thereby differentiating between the constant light absorbers (i.e., skin, tissue, and venous blood) and the alternating light absorbers (i.e., arterial blood flow). In certain embodiments sensor 22 determines the detected values of greater light absorption as a percentage of the values of lesser light absorption, and compares the percentage value to a look-up table to determine at least one of an SpO2 and a COHb level.

Sensor 22 may be coupled to transmitter 12 using cabling, wiring, a flat harness, or another known method such as with low noise cables (‘LNC’) manufactured by Masimo corporation (‘LNC’ is a trademark of the Masimo corporation). In certain embodiments, in addition to sensor 22 acting as a pulse oximeter, it may also concurrently act as a heart rate sensor. In certain embodiments as a heart rate sensor, sensor 22 derives a number of heart beats by detecting the periods of greater light absorption in relation to the periods of lesser light absorption, as explained above. In certain embodiments, sensor 22 may also act as a sensor that provides a peripheral perfusion index.

Also shown in FIG. 2 is sensor 23 coupled to head band 24. In certain embodiments sensor 23 may act as a sensor for a peripheral perfusion index. In certain embodiments sensor 23 may comprise a temperature sensor, a heart rate sensor, an environmental carbon monoxide detector, or another type of sensor.

In certain embodiments where either of sensors 22 and/or 23 act as a peripheral perfusion index sensor, the pulsative component of arterial blood flow may be monitored for amplitude as a percentage of the amplitude of the non-pulsative component as derived from the amount(s) of infrared light absorbed, to thereby calculate peripheral perfusion. By comparing the amplitude of the non-pulsative component of arterial blood flow to the amplitude of the pulsative component over time, a percentage (and/or a ratio) may be determined that may then be compared to values in a look-up table that enables conversion of the percentage (and/or ratio) to an accurate peripheral perfusion index. For instance, when peripheral hypo-perfusion exists, the pulsative component decreases substantially, and because the non-pulsative component does not change, the ratio between the two components decreases, allowing for an accurate estimation of a peripheral perfusion index value.

In certain embodiments, sensors 22 and/or 23 may comprise a memory (e.g., a read-only memory) that stores a look-up table, for example any of the look-up tables discussed above, and may therefore communicate data comprising an SpO2 value, a carboxyhemoglobin value, and/or a peripheral perfusion index value to transmitter 12. In certain embodiments, sensors 22 and/or 23 may comprise a processor. In certain embodiments, sensors 22 and/or 23 may be coupled directly and/or indirectly to at least one of a transmitter, a processor, and a receiver. In certain embodiments, a transmitter, a processor, a receiver, an output device, and/or a sensor may be coupled with at least one other of the named components. Further, ‘coupled,’ as used herein, may be used to describe a wired or wireless connection for communicating and/or otherwise conveying raw data and/or processed data (or information). Wireless coupling may include an electromagnetic transmission to include radio frequency, photonic, laser, or other transmission. Wireless coupling may also include ultrasonic or ultrasound transmissions. Wired coupling may include wires, busses, fiber optics, or other transmission. In certain embodiments, sensors 22 and/or 23 may not comprise a memory, and may therefore communicate a raw red-to-infrared light ratio and/or a raw pulsative-to-non-pulsative percentage/ratio to transmitter 12 wherein the raw values are either compared to a look-up table in the transmitter or the raw values are transmitted to a receiver for comparison to a look-up table at the receiver. In certain embodiments, the raw values may be converted to useable data using a conversion matrix, an algorithm, or other known method for data conversion.

In certain embodiments sensor 23 comprises an environmental carbon monoxide detector. As such, sensor 23 may comprise any of a biomimetic, an electro-chemical, and/or a semiconductor sensor. As a biomimetic sensor, sensor 23 may comprise a chem-optical or gel cell sensor that works with a form of synthetic hemoglobin. In the presence of carbon monoxide the synthetic hemoglobin darkens. Outside the presence of carbon monoxide the synthetic hemoglobin lightens. A photo-receptor may be used to record the appearance (i.e., relative darkness or lightness) of the synthetic hemoglobin, and then a look-up table may be consulted to derive an environmental carbon monoxide level.

In certain embodiments sensor 23 may comprise an electrochemical sensor, such as a small fuel cell. As carbon monoxide is detected, the sensor produces a current amplitude that corresponds to the level of carbon monoxide in the atmosphere. The electrochemical cell may include two electrodes, connection wires and an electrolyte. A typical electrolyte would be a small amount of sulfuric acid. Environmental carbon monoxide is oxidized at one electrode, and converted to carbon dioxide. At the other electrode, oxygen is consumed. Such electrochemical cells are typically highly accurate with a linear output representing carbon monoxide concentration. An electrochemical cell requires minimal power, operates at typical room temperatures, and typically has a reusable lifetime of many years.

In certain embodiments sensor 23 may comprise a semiconductor carbon monoxide detector. In such an embodiment, thin wires of semiconductor tin dioxide are placed on an insulating ceramic base to provide a sensor monitored by an integrated circuit. Environmental carbon monoxide reduces resistance across the tin dioxide, resulting in a electrical current value. The current value is converted by use of a look-up table to an accurate estimation of detected carbon monoxide.

Sensor 23 is communicatively coupled with transmitter 12. Data detected by sensor 23 is communicated to transmitter 12 for wireless transmission to a remotely located receiver.

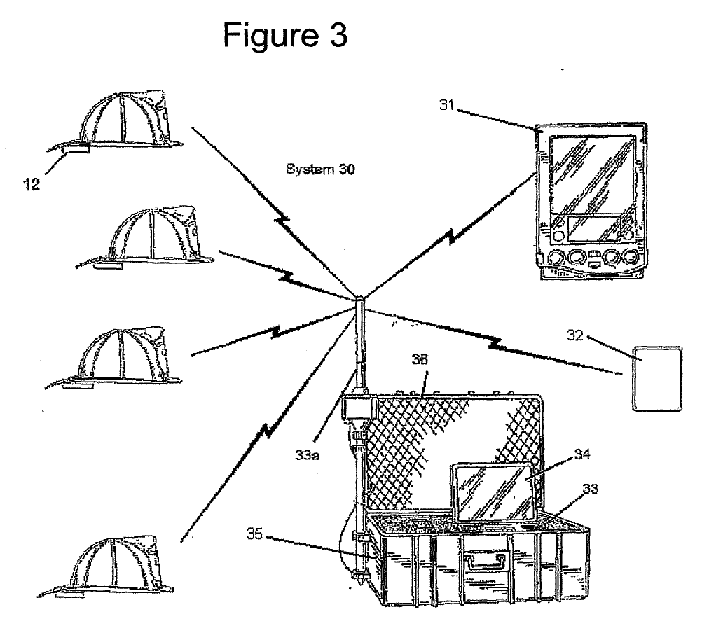

FIG. 3 illustrates certain embodiments including a system 30. System 30 comprises a plurality of personnel equipment 11. The plurality of personnel equipment 11 each individually include at least one sensor, for example, one of the above-described pulse oximeter, peripheral perfusion, heart rate, and/or environmental carbon monoxide sensors. The plurality of personnel equipment 11 each individually comprises a transmitter 12. In certain embodiments, an individual transmitter 12 operates by receiving data or information from the at least one sensor and then wirelessly communicating that data or information to a receiver located remotely from the user equipment 11, such as receiver unit 35 that comprises part of system 30. The system 30 may further comprise a signaling device 31 (such as a Personal Digital Assistant, or PDA), and/or a database server that is remotely located from the system 30. In certain embodiments, system 30 comprises a display 34, a processing unit 33, a carrying case 36, and an antenna 33a.

In certain embodiments, a sensor or sensors coupled with personnel equipment 11 detect(s) physiological and/or environmental conditions for a plurality of emergency responders. Data comprising the physiological and/or environmental conditions is communicated to transmitter(s) 12. Transmitter(s) 12 wirelessly communicate the data to receiving unit 35 via antenna 33a. Transmission may be accomplished by any of CDMA, TDMA, FDMA, or other well-known methods for wirelessly transmitting data and information.

In certain embodiments, receiving unit 35 may comprise a carrying case 36, a processing unit 33, an antenna 33a, and/or a display 34. Processing unit 33 may comprise a conventional processor or microprocessor, a hard drive, read-only and/or random-access memory, a number of input devices such as a keyboard and/or a mouse, a number of output devices such as a printer and/or a display 34, and processor-executable instructions stored in either or both of the hard drive and/or memory. Processing unit 33 may comprise a laptop computer. Receiving unit 35 may comprise a transceiver, capable of both transmitting and receiving from and to transmitter(s) 12, signaling device 31, and/or a database server 32. Signaling device 31 may comprise a Personal Digital Assistant, and database server 32 may comprise a database of permissible exposure limit values and/or material safety data sheets for various chemicals and/or gases.

The processor-executable instructions stored within processing unit 33 may be computer and/or machine readable instructions, and may comprise various look-up tables, such as those described above useful for determining levels of oxygenated and deoxygenated hemoglobin and/or carboxyhemoglobin, peripheral perfusion, and/or environmental carbon monoxide levels. The processor-executable instructions may also comprise a database of permissible exposure limit (PEL) values for various chemicals and/or gasses, such as those promulgated by the Occupational Safety and Health Administration (OSHA), the National Institute for Occupational Health and Safety (NIOSH), or similar organizations or government entities. The database may comprise limit values for both acceptable and unacceptable levels of exposure to carbon monoxide, for example, limit values provided by OSHA, NIOSH, or another organization.

Certain embodiments comprise limit values for both acceptable and unacceptable exposure levels as provided by OSHA. OSHA provides that exposure limits of less than 50 parts-per-million over a time-weighted average of 8 hours may be acceptable. OSHA recommends a maximum allowable 8-hour exposure of 35 ppm for carbon monoxide, which corresponds to an expected carboxyhemoglobin level of approximately 5 percent. Exposure at the PEL of 50 ppm for 8 hours is expected to yield a carboxyhemoglobin level of 8 to 10 percent in most healthy, non-smoking individuals. The current OSHA permissible exposure limit (PEL) for carbon monoxide is 50 parts per million (ppm) per parts of air (55 milligrams per cubic meter (mg/m(3)) at an 8-hour time-weighted average (TWA) concentration.

Certain embodiments comprise limit values for both acceptable and unacceptable exposure levels as provided by NIOSH. NIOSH has established a recommended exposure limit (REL) for carbon monoxide of 35 ppm (40 mg/m(3)) at an 8-hour TWA and 200 ppm (229 mg/m(3)) as a ceiling.

Certain embodiments comprise limit values for both acceptable and unacceptable exposure levels as provided by the American Conference of Governmental Industrial Hygienists (ACGIH). ACGIH has assigned carbon monoxide a threshold limit value (TLV) of 25 ppm (29 mg/m(3)) at a TWA for a normal 8-hour workday and a 40-hour workweek.

Any or all of the preceding values for carbon monoxide exposure may comprise at least a part of a database within processing unit 33. In certain embodiments, the database may be located externally to processing unit 33, such as in a flash memory drive, an external hard drive, or at a location remote from the receiving unit 35 such as the case where database server 32 is in wireless communication with receiving unit 35 and processing unit 33.

Receiving unit 35 receives physiological and/or environmental data from at least one of the plurality of transmitters 12 coupled to the personnel equipment 11. Data transmitted by transmitters 12 may comprise location information, such as GPS information. Any data that has not been previously converted to a detected carboxyhemoglobin value, to a peripheral perfusion index value, to a heart rate value, and/or to an environmental carbon monoxide value, may be converted to such by use of a look-up table, a conversion algorithm, or another well-known method by processing unit 33. The converted data (comprising at least one of a detected carboxyhemoglobin value, a peripheral perfusion index value, a heart rate value, and/or an environmental carbon monoxide value) is recorded within processing unit 33, for instance to a hard drive, a Readable/Writable CD, an optical drive, or another type of computer-readable and/or machine-readable medium. The converted data may be stored within a matrix and/or a spreadsheet that plots the converted data over a time period. Transmitter(s) 12 may send a continuous or intermittent stream of data to receiving unit 35, wherein the receiving unit 35 communicates the data to processing unit 33. Processing unit 33 may then record samples of the converted data, for example, at five second intervals for a sampling period of thirty seconds or more.

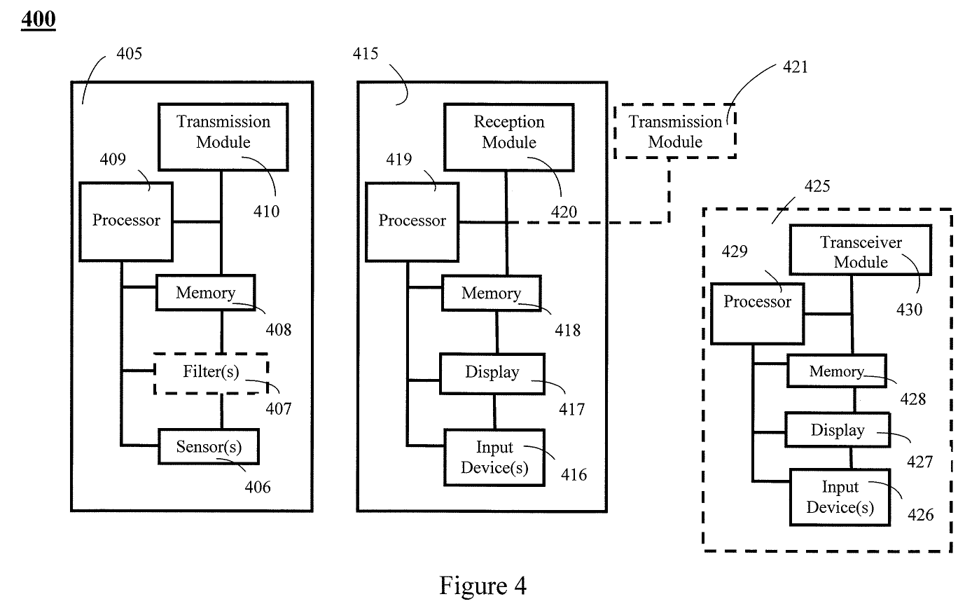

FIG. 4 is a block diagram of a certain embodiment of a physiological monitoring system 400 comprising a transmitter unit 405, a receiver unit 415, and an optional signaling device unit 425. As shown in FIG. 4, the transmitter unit 405 comprises sensor(s) 406, optional filter(s) 407, memory 408, processor 409, and transmission module 410. The receiver unit 415 comprises input device(s) 416, display 417, memory 418, a processor 419, reception module 420, and optional transmission module 421. Optional signaling device 425 comprises input device(s) 426, display 427, memory 428, a processor 429, and a transceiver module 430.

The block diagram for transmitter unit 405 provides an example of the basic components that may be included as a part of a transmitter or transmitters attached to personnel equipment, for example, the transmitter 12 shown in FIGS. 1 and 3. As shown in FIG. 4, the transmitter unit comprises a sensor or sensors 406. In certain embodiments, sensor or sensors 406 may comprise any number of sensors that may be used to monitor at least one physiological and/or environmental condition for an emergency responder, for example, any of the previously described sensors useful for monitoring carboxyhemoglobin, a peripheral perfusion index, heart rate, and/or environmental carbon monoxide, or similar sensors. While shown as a part of transmitter unit 405, sensors 406 may be located remotely from, but in communicative contact with, transmitter unit 405. For example, sensors 406 may be located in various locations of an emergency responder's personnel equipment, whether a helmet, protective eyewear, or another location. In certain embodiments, the output from sensors 406 may be provided to a filter and/or a filter bank 407. Filter and/or filter bank 407 may filter the output of sensors 406 for ambient noise and/or artifact. Data from sensor 406 may be stored or logged into memory 408.

In certain embodiments, memory 408 is at least one of volatile and non-volatile memory. For instance, memory 408 may comprise a volatile random access memory coupled with a read-only memory. The read-only memory may store a look-up table, for example, with values for converting a raw sensor value to an accurate carboxyhemoglobin and/or environmental carbon monoxide value. A sensor value from sensors 406 may be written to random access memory, and then processor 409 may instruct that a comparison be made between the value written in random access memory to a value in a look-up table in read-only memory to derive an accurate carboxyhemoglobin and/or environmental carbon monoxide value.

In certain embodiments, transmitter unit 405 comprises a reception module (not shown) for receiving information sent from receiver unit 415. In certain embodiments, transmitter unit 405 comprises an alerting module (not shown) that provides at least one of an audio, visual, and/or tactile feedback to the emergency responder, such as feedback comprising safety information on at least one of time of remaining safety in the region, a warning to exit the region, an indication of continued safety in the region, and/or an indication of impending danger (as such is determined by receiver unit 415 based on a processing of the values of levels for at least one of carboxyhemoglobin, a peripheral perfusion index, heart rate, temperature, ambient CO levels, and/or other detected data). Tactile feedback may comprise use of at least one of a vibratory stimulator and a electric shock stimulator. Visual feedback may comprise use of at least one of an LED, LCD, monitor display, etc... Auditory feedback may comprise use of at least one of an earphone, a headset, earbuds, speakers, speaker phones, a handset, or other type of feedback device.

In certain embodiments, any of processors 409, 419, and/or 429 may be standard microprocessors capable of executing computer-readable and/or machine-readable instructions, for example, software instructions stored in any of memories 408, 418, and/or 428. Processor 409 may compute and process threads provided from any of sensor(s) 406, filter(s) 407, memory 408, and/or transmission module 410. Processor 419 may compute and process threads provided from any of input device(s) 416, display 416, memory 418, reception module 420, and/or transmission module 421. Processor 429 may compute and process threads provide from any of input device(s) 426, display 427, memory 428, and/or transceiver module 430.

In certain embodiments, transmission module 410 wirelessly communicates data received from at least one of memory 408 and sensor(s) 406 to receiver unit 415 using an antenna (not shown). Transmission module 410 may operate under various wireless communication methods, for example, a CDMA, TDMA, a FDMA method, or another known method. Transmission module 410 may transmit the data received from at least one of memory 408 and sensor(s) 406 in packets to receiver unit 415. Data may be transmitted in a continuous stream or intermittently.

Receiver unit 415 comprises input device(s) 416, display 417, memory 418, a processor 419, a reception module 420, and an optional transmission module 421. Receiver unit 415 may comprise a laptop computer coupled with a wireless networking card and/or coupled to a networking cable. Receiver unit 415 wirelessly receives the data transmitted by transmitter unit 405 at reception module 420. Reception module 420 comprises an antenna (not shown) and may comprise any components useful for receiving data, based on the type of transmission scheme employed. For instance, if a CDMA method is employed, reception module 420 may comprise a number of correlators and queues for demodulating spread spectrum data, as would be understood by one of skill in the art.

Receiver unit 415 comprises input device(s) 416. Input devices may include any of a keyboard, mouse, touch screen, flash drive, or another type of input device. Display 417 may be a touch-screen, a flat screen, and/or another display. Display 417 is in communicative contact with the input device(s) 416, memory 418, and processor 419. Data received from transmitter unit 405 is received and initially demodulated at reception module 420. Demodulated data is then stored in memory 418. Memory 418 may comprise any type of computer-readable and/or machine-readable media. For instance, memory 418 may comprise a hard drive coupled with random access memory and/or read-only memory. Memory 418 may comprise an optical drive and/or flash memory, or other non-volatile and/or volatile media.

In certain embodiments, memory 418 comprises any number of look-up tables for determining values of carboxyhemoglobin, environmental carbon monoxide levels, peripheral perfusion index values, or for other detected data (individually or collectively, the “acquired data”). In certain embodiments, memory 418 stores computer-readable and/or machine-readable instructions that are capable of being executed by processor 419. For instance, memory 418 may store instructions for processor 419 that comprise a trending and/or analysis of the acquired data received from transmitter unit 405.

As used herein, ‘trending,’ or ‘trend’ is used to describe any form of data processing, and specifically includes a regression function with respect to time. In certain embodiments, ‘trend,’ and/or ‘trending’ may comprise a determination of an attribute of the regression function, for instance, at least one of a derivative and an integral. In certain embodiments, ‘trending’ and/or ‘trend’ may comprise an extrapolation of a regression function for a future value. A derivate and/or integer may be extrapolated to derive a forecast of future expected values such as carboxyhemoglobin and/or environmental carbon monoxide levels for a particular emergency responder (or responders) that is (are) being wirelessly monitored. The trending and/or analysis may comprise an analysis of singular and/or multiple physiological and/or environmental values to provide feedback to a user. For example, processor 419 may execute instructions stored in memory 418 for a comparison of carboxyhemoglobin levels with detected levels of environmental carbon monoxide to create feedback that is then provided to a user, the feedback comprising information on at least one of time of remaining safety in the region, a warning to exit the region, an indication of continued safety in the region, and/or an indication of impending danger. For instance, the feedback may comprise information indicating that a CO threshold has been exceeded for a particular length of time, for instance, a detected level of 35 ppm CO for an hour or greater may indicate that the person has an hour (or other period of time) of continued safety in the region. A detected level of 100 ppm CO may trigger a warning to exit the region. A detected level of 20 ppm CO may indicate continued safety in the region. And a detected level of 200 ppm CO may indicate impending danger and the need to leave the region.

In certain embodiments, a trend of acquired data may include a determination of expected levels of carboxyhemoglobin present in the person being monitored based on detected levels of environmental carbon monoxide. In certain embodiments, the determination of expected COHb levels may comprise application of the Coburn-Forster-Kane equation to the detected levels of ambient CO, such that:

% expected COHb=(3.317×⁻⁵) (ppm CO)^{1.036 (RMV) (t)}, where

ppm CO=ambient carbon monoxide levels in parts per million;
RMV =an expected respiratory minute volume of air breathed by the at least one emergency responder in liters per minute; and
(t)=exposure time for the at least one emergency responder in minutes.

On average, a healthy adult person at rest or under light physical exertion may be expected to have an RMV value of between 5-8 liters per minute. Under strenuous activity the same person may be expected to double their resting RMV value.

Receiving unit 415 may wirelessly couple to an external database (for example, element 32 in FIG. 3) for downloading permissible exposure limit (or threshold limit) values, and/or for downloading material safety data sheet information. Multiple receiving units 415 may connect to an external database where data and information may be stored, analyzed, retrieved, and evaluated by a regional command that oversees a plurality of command posts, where each individual command post comprises at least one receiving unit 415. Receiving unit 415 may comprise an optional transmission module 421 for transmitting data to either of transmitter unit 410 and/or signaling device 425.

Signaling device 425 may be a PDA or other portable device capable of receiving information transmitted by the transmission module 421 from receiver unit 415. Signaling device 425 comprises input device(s) 426 that may be a voice input device, a keyboard, a touch screen, or another input device. Display 427 may display alarms, alerts, and/or provide feedback values received from receiver unit 415 (such as such as feedback comprising information on at least one of time of remaining safety in the region, a warning to exit the region, an indication of continued safety in the region, and an indication of impending danger). Memory 428 may be either volatile and/or non-volatile memory capable of storing information and/or instructions. Processor 429 may be a microprocessor capable of executing computer-readable and/or machine-readable instructions stored in memory 428. Transceiver module 430 may comprise any number of modulators and demodulators, correlators, queues, or other components depending upon the type of wireless communication scheme employed, as would be understood by one of skill in the art.

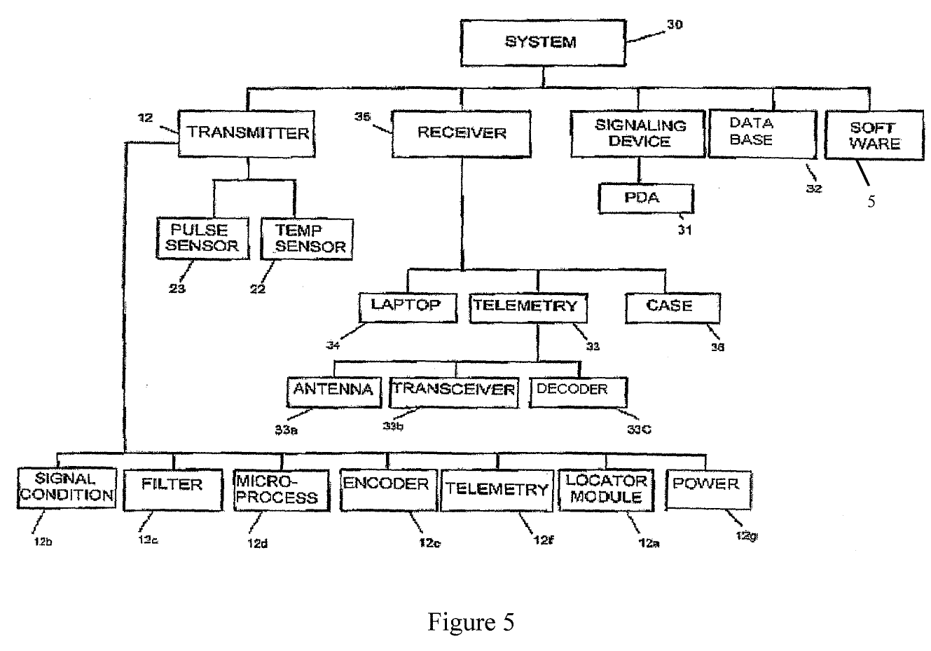

FIG. 5 is a block diagram of certain embodiments including a carboxyhemoglobin reporting system. As shown in the figure, system 30 comprises transmitter(s) 12, receiver 35, signaling device / PDA 31, database 32, software 5, pulse sensor 23, temperature sensor 22, laptop 34, telemetry 33, case 36, antenna 33a, transceiver 33b, decoder 33c, signal conditioner 12b, filter 12c, microprocessor 12d, encoder 12e, telemetry 12f, locator module 12a, and power supply 12g. The system 30 actively monitors location and physiological data that is detected and transmitted from one or more persons (e.g., firefighters fighting a fire). Data is acquired at sensors 23 and 22. Sensors 23 and 22 may comprise pulse oximeter sensors, temperature sensors, peripheral perfusion index sensors, heart rate sensors, location sensors (such as GPS sensors), or other sensors (individually or collectively, the “acquired data”). The acquired data is transmitted from the individual person(s) being monitored to a receiving unit 35. The receiving unit 35 comprises a machine-readable media that stores the acquired data.

Since most firefighter activities typically involve multiple personnel, system 30 is configured to simultaneously measure, record, and transmit acquired data for a range of persons, from one person to a plurality of people, including an excess of 100 persons. In certain embodiments the system 30 is employed where the transmitter(s) 12 is/are coupled to a helmet, for instance a firefighter's helmet or a military helmet. In certain embodiments system 30 is employed where the transmitter(s) 12 is/are coupled to other clothing, equipment, and/or emergency responder gear. The system 30 is configured to be employed when either the persons being monitored are engaged in physical activity and/or when the persons being monitored are at a reduced activity level. Monitoring when at a reduced activity level may provide baseline values and/or allow for monitoring of undesirable changes in physiology after the physical activity has been completed.

The transmitter(s) 12 process the acquired data from sensors 22 and 23. The acquired data is then transmitted to the receiver 35, which may be located at a command post. The processing performed by transmitter(s) 12 may comprise conditioning and filtering of the acquired data, encoding of the acquired data, and transmission of the acquired data to receiver 35. Simultaneous transmissions by multiple transmitters 12 may be accomplished using time division, code division, and/or frequency division multiple access, as one of skill in the art would comprehend. The acquired data may be sent using unique coded identifiers that enable receiving unit 35 to decode information from various transmitting units 12. Signaling device (PDA) 31 may comprise multiple PDA units that enable a supervising user (or multiple supervising users) to monitor and track the location and physiological condition of the person(s) being monitored. In certain embodiments, the PDA/signaling unit(s) comprise user-selected fields to allow for searching and/or particularized data display, for instance, data on particular person(s) being monitored and/or levels of COHb being detected in the person(s) being monitored.

Receiving unit 35 receives the acquired data from transmitter(s) 12 and processes the data to provide feedback to users. In certain embodiments, the user(s) may be the person(s) being monitored. In certain embodiments, the user(s) may be supervisor(s) remotely located from the person(s) being monitored. In certain embodiments, the receiver unit 35 may be located at a command post, and may comprise a portable processing unit, such as a laptop or other portable computer. In certain embodiments, the receiver unit 35 comprises a display, a telemetry unit 33. In certain embodiments, the telemetry unit 33 receives the acquired data wirelessly transmitted by transmitter(s) 12. In certain embodiments, the receiving unit 35 comprises a machine-readable media for storing the acquired data for subsequent analysis and processing. In certain embodiments, the receiving unit 35 is portable, i.e., is capable of being transported by one or more persons when stored within a case 36.

In certain embodiments, telemetry element 33 includes an antenna 33a, a transceiver 33b, and an encoder 33c. In certain embodiments, the telemetry unit 33 decodes encoded signal comprising the acquired data sent from transmitter(s) 12. In certain embodiments, the receiving unit 35 recognizes a coded identifier providing by each transmitter 12 and organizes results of the acquired data for each person being monitored. In certain embodiments the receiving unit 35 comprises a machine-readable (and/or computer-readable) media that stores acquired data. In certain embodiments, the receiving unit 35 is equipped with software that includes management information and enable communication with a centralized database 32. In certain embodiments the centralized database may comprise accumulations of data, for instance, threshold limit values, such as those values discussed above in relation to OSHA and/or NIOSH limit values.

In certain embodiments the receiving unit 35 receives encoded signals sent from the transmitting unit(s) 12 and processes the acquired data contained within the encoded signals to allow for calculating results for use by an end-user, such as the personnel and/or supervisors that at a command post. When the acquired data includes data that exceeds a predetermined level, the receiving unit 35 may display feedback alerting a user that the level has been exceeded. For instance, if a threshold for CO is set at 50 ppm, and the acquired data indicates that at least one person being monitored has entered a region where the ambient CO exceeds 50 ppm, that data is then transmitted to receiving unit 35, which then communicates that the threshold has been exceed by at least one of displaying the exceeded limit on a display, creating an audible alert, creating a tactile alert, and/or sending the exceeded limit to signaling device 31 with any of a visual, audible, and/or tactile alerts to notify that the threshold has been exceeded. In certain embodiments, the feedback regarding the exceeded threshold comprises the name, measured result, time, geography, and/or identifier of the person being monitored. The feedback may be provided to the person being monitored with at least one of audible, visual, and tactile feedback. The person in question may be alerted, removed from the region, and evaluated.

In certain embodiments, the signaling device 31 and laptop 34 communicate with the receiving unit 35, and alert the command post personnel or other personnel when data is outside of acceptable limits. In certain embodiments, the signaling device may be a pager, a PDA, or other portable electronic device (such as a cell phone), that is capable of communicating and alerting a user to possible alarms sent by transceiver 33b. In certain embodiments the signaling device 31 may be worn or held, and may vibrate, produce audible noise, and/or produce visual feedback when a suspect event (e.g., when a threshold limit value is exceeded) to alarm a user about events as they transpire. Acquired data from transmitters 12 is detected, measured, and recorded. Acquired data may comprise a change in physiological status of the personnel being monitored, a change in body temperature, pulse, decreased oxygenation, a decreased peripheral perfusion index, along with times of the incident(s) and other data related to incident management (e.g., location, possible trends among pluralities of monitored persons, etc . . . ).

In certain embodiments system 30 comprises a server 32, such as a database server. The database may store data from a plurality of remote sites, each having their own system 30 for monitoring multiple persons. The database 32 may be Internet-capable, allowing all users a common methodology for connectivity and communication, as one of skill in the art would comprehend.

In certain embodiments system 30 may be configured to adjust monitoring, sensitivity, and/or calculations based upon various inputs. For instance, system 30 may be changed from a configuration where a first threshold limit value alarms at 30 ppm CO to where the threshold limit value alarms at 50 ppm CO. Additionally, system 30 may be configured to provide different charts and comparisons of acquired data, for instance a chart that originally simply showed detected COHb levels may be configured to show both COHb in the person(s) and detected ambient CO levels in relation to each other, with an alarm set at a predetermined level that comprises a combined value of detected ambient CO and detected COHb in the person(s). Thus, the operation parameters and standards of system 30 components, such as the transmitter(s) 12, the receiving unit(s) 35, and the signaling device(s) 31 may be manipulated and adjusted for application-specific monitoring and/or the adjust for each individually monitored person(s) recent history and data. For instance, monitored persons who are learned to be smokers may have a different COHb monitoring level threshold than non-smokers. Additionally, those recently exposed to amounts of CO may be considered more sensitive to additional exposure, and may have a lower threshold limit value for ambient CO and/or detected levels of COHb.

Referencing FIG. 3 and machine-readable (and/or computer-readable) instructions as mentioned herein, such instructions (e.g., those stored within processing unit 33) may comprise processor-executable instructions to trend the acquired data over a sampling period (or over multiple sampling periods). For example, sensor 22 at time zero for fire fighter John may initially indicate that John possesses a carboxyhemoglobin level of 0.5%. Sensor 23 may initially indicate at time zero that fire fighter John's breathing environment comprises a 15 PPM level of carbon monoxide. The data for time zero is transmitted by transmitter 12 to receiving unit 35. Processing unit 33 records this data and awaits further data to continue analyzing the acquired data.

Simultaneously, fire fighter Jane may have had a carboxyhemoglobin reading from her sensor 22 at time zero of 1% carboxyhemoglobin, and an environmental carbon monoxide level of 30 PPM. A supervising fire fighter (i.e., a user of the receiving unit 35) may remotely monitor fire fighters Jane and John by observing a display 34 that comprises graphs, charts, fields, and/or maps that may comprise approximately real-time data for analysis, for example, trending values of carboxyhemoglobin, a peripheral perfusion index, temperature, heart rate, and/or environmental carbon monoxide.

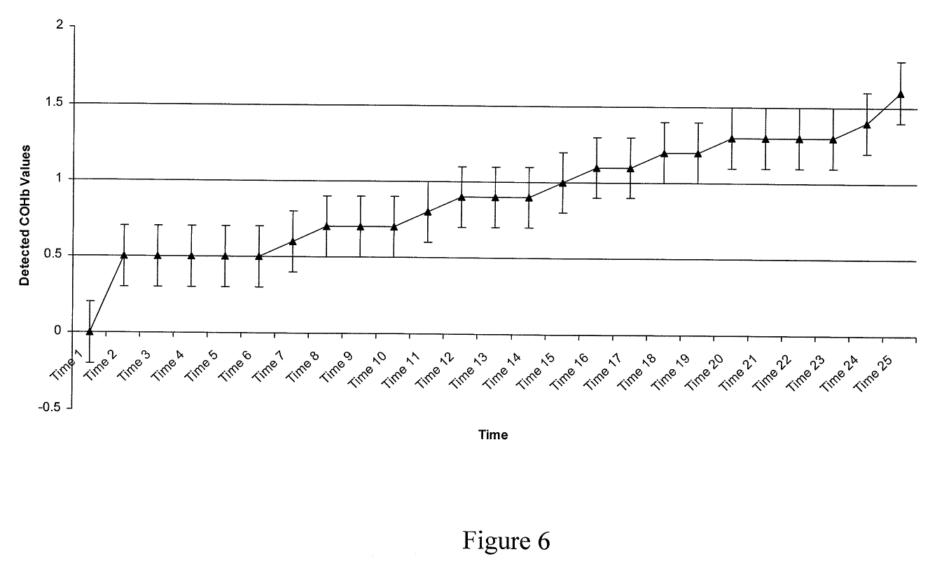

In certain embodiments, processing unit 33 may receive and record multiple values of carboxyhemoglobin and environmental carbon monoxide over a sampling period, may extrapolate a trend in the values, and provide a user with feedback on the trend. For example, as shown in FIG. 6, fire fighter Jane has been wirelessly monitored for detected levels of carboxyhemoglobin over a sampling period comprising 20 discrete sampling periods (the chart shows 25 periods, but period 21-25 are an extrapolation of the previous 20). The data shown has undergone a regression function, as one of skill in the art would comprehend for statistical analysis. In certain embodiments the regression function may be a derivative function, and in certain embodiments the regression function may be an integral function.

As shown in FIG. 6, the level of detected carboxyhemoglobin has risen from approximately zero percent at time period 1 to just under one percent at time period 15, and then has risen fairly steadily to approximately 1.3 percent at time period 20. By extrapolating the regression function, the chart provides expected future values of Jane's COHb in time periods 21-25. As shown during those periods, Jane's COHb is expected to exceed 1.5% by the end of the 25th time period. With this information, a supervising fire fighter at a command center may evaluate the trend, and perhaps based on available human resources, decides to pull fire fighter Jane from the fire fighting line so that her rise in COHb levels may be reversed either through application of fresh air and/or application of masked oxygen of greater than 21%.

In certain embodiments, a preset level of either carboxyhemoglobin and/or detected environmental carbon monoxide triggers a ‘soft limit’ within processing unit 33 that alerts at least the supervising fire fighter (i.e., a user of receiving unit 35) with visual, tactile, and/or audible feedback that the soft limit has been reached. An example of a soft limit preset level may be a detected level of environmental carbon monoxide that meets or exceeds 10 PPM for at least two adjacent time periods. Another example of a soft limit preset level may be a detected level of carboxyhemoglobin that meets or exceeds 0.5% in any time period. The soft limit audible and/or visual feedback may comprise a requirement that the user acknowledge the feedback, for instance by using a mouse to click on a radio button that then ends or limits the amount of audible, tactile, and/or visual feedback. In certain embodiments, the soft limit may include feedback that informs the user of a remaining time of safety in the region, or an indication of continued safety in the region.

In certain embodiments, the processing unit 33 may execute instructions to apply a regression function to any of the received data. For instance, a derivate, integral, or other function.

In certain embodiments, a preset level of either carboxyhemoglobin and/or detected environmental carbon monoxide triggers a ‘hard limit’ within processing unit 33 that alerts the supervising fire fighter (i.e., a user of receiving unit 35) with visual and/or audible feedback that the hard limit has been reached. An example of a hard limit preset level may be a detected level of environmental carbon monoxide that meets or exceeds an OSHA, NIOSH, or other determined safety limit value. For instance, a hard limit may be set at a time weighted average of 25 to 35 PPM for an expected period of 8 hours. Another example of a hard limit preset level may be a detected level of carboxyhemoglobin that meets or exceeds a value of somewhere between 2.5% and 5% in any time period. In certain embodiments, a hard limit may trigger feedback comprising an indication to the user for the person to exit the region, and/or an indication of impending danger.

In certain embodiments, hard limit audible, tactile, and/or visual feedback may comprise a requirement that the user acknowledge the feedback, for instance by using a mouse to click on a radio button that then ends or limits the amount of audible and/or visual feedback.

In certain embodiments, at time period 20, because the regression function has estimated that at time period 25 a level of greater than 1.5% carboxyhemoglobin will be reached, processing unit 33 may provide the user of receiving unit 35 with visual, tactile, and/or audible feedback that a soft limit is expected to be reached at time period 25. In certain embodiments, the processing unit 33 may provide the user with visual, tactile, and/or audible feedback only upon actually reaching an actual hard limit, e.g., 2.5% COHb. In certain embodiments the visual, tactile, and/or audible feedback for either of an expected hard limit or an actual hard limit comprises a persistent audible alarm and/or a flashing notice on display 34. For example, the name of the monitored emergency responder may flash in alternating bright colors from black to red and back again. In certain embodiments, the receiving unit 35 transmits the hard limit alarm to one or more of the wirelessly monitored emergency responder personnel and/or to the signaling device 31 along with the name or an identifier of the emergency responder whose hard limit has been reached. For instance, in certain embodiments the expected or actual hard limit may be transmitted to any or all of the wirelessly monitored emergency personnel to provide them with audio and/or visual information on which emergency responder has reached a hard limit.

In certain embodiments, each or either of the soft and hard limits may correspond to any of the physiological and/or environmental values that are detected by sensor 22 and/or sensor 23. In certain embodiments, sensor 22 may provide data on carboxyhemoglobin, a peripheral perfusion index, and/or heart rate, and sensor 23 may provide data on environmental carbon monoxide levels, with all of the noted values being received and recorded by receiving unit 35 for subsequent trending and analysis.

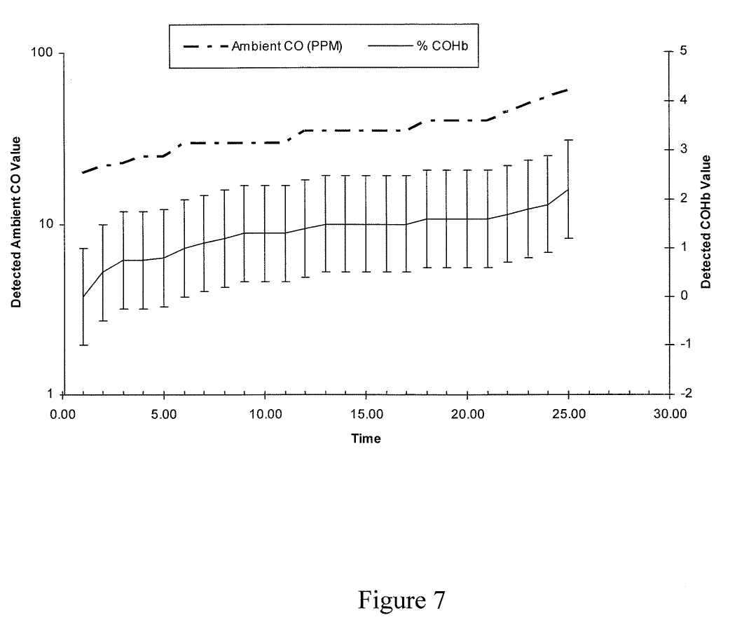

In certain embodiments, the processing unit 33 may execute instructions to apply a regression function to any of the received data. For instance, as shown in FIG. 7, a regression function has been applied to fire fighter John's detected levels of carboxyhemoglobin, as reflected by the solid line labeled ‘% COHb.’ As shown in FIG. 7, the regression function comprises a curved slope from time period zero to time period 20 and continuing as an extrapolation from time period 25 to time period 30. From time periods zero through 20, John's detected levels of carboxyhemoglobin have risen steadily from approximately 0% at time period zero to approximately 1.5% at time period 20. An extrapolation of the regression function provides that at time period 25 John's level of expected COHb will be greater than 2%.

In certain embodiments, at time period 5, a soft limit of 1% detected carboxyhemoglobin occurs, and the processing unit 33 has alerted at least the user of receiver unit 35 to that fact. The regression function applied to John's detected level of carboxyhemoglobin shows a steady change in rate of rise beginning at about time 5 and continuing to rise through time period 20.Processing unit 33 alerts with a hard limit alarm by providing audio, tactile, and/or visual feedback to the user of receiving unit 35 (for example, a supervising fire fighter). In certain embodiments, the feedback may also be communicated to at least one of signaling device 31 and/or to any or all of the emergency responders being wirelessly monitored. The feedback may comprise the name and/or identifier of the emergency responder who has reached the preset hard limit and/or other information such as the value of the level reached and/or geographic location of the person(s) being monitored.

It is envisioned that the soft limit(s) discussed herein (such as when the feedback discussed above comprises information of a time of remaining safety in a region, and/or an indication of continuing safety in the region) apply to a first threshold that indicates at least one emergency responder is within a geographic zone of danger that is acceptable. ‘Acceptable’ is meant to reflect a range of values that a person may safely operate within for a period of time. For example, a healthy human adult is expected to safely work within an environment of 25 to 35 ppm CO over an time weighted average of 8 hours (according to NIOSH and OSHA data). It is envisioned that the hard limit(s) discussed herein apply to a second threshold that indicates at least one emergency responder is within a physiological and/or environmental zone of danger that is unacceptable. ‘Unacceptable’ is meant to reflect a range of values that a person may not typically safely operate within except for possibly a short period of time, for instance, those cases where the feedback would inform the user of a warning to exit the region and/or an indication of impending danger. For example, a healthy human adult is expected to survive a short exposure to 200 PPM CO (according to NIOSH data) so long as that value is not exceeded and so long as an 8 hour time weighted average does not exceed a recommended exposure limit of 35 ppm. For example, in certain embodiments a soft limit value may be set for any exposure to carbon monoxide, and a hard limit value may be set for a range of carboxyhemoglobin of between 2.5 and 3.5% for instance, or to an exposure of greater than 200 PPM carbon monoxide, or to an exposure of between 100 and 200 PPM carbon monoxide for any two time periods within a prescribed time frame, for instance, 3 to 5 minutes.

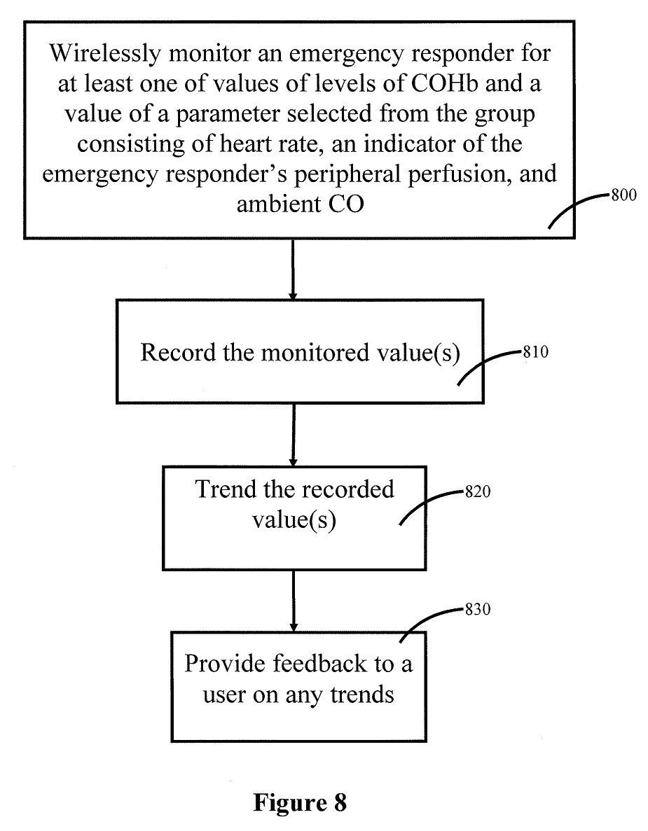

FIG. 8 illustrates certain embodiments that encompass the above-described embodiments as a method and/or as instruction steps. At step 800, an emergency responder is wirelessly monitored for at least one of values of levels of COHb and values of levels selected from the group consisting of heart rate, peripheral perfusion, and environmental CO. In certain embodiments, any number of emergency responders may be wirelessly monitored as part of step 800, including with a number of sensors, transmitters, and receivers, for example, those sensors, transmitters, and/or receivers described previously. In step 810, the monitored values are recorded, for instance to a computer-readable and/or machine-readable medium. In certain embodiments, the monitored values may be stored in a laptop computer comprising a hard drive and/or a memory. In step 820, the recorded values undergo trending and analysis, as discussed above, for instance with a regression function. In certain embodiments, the trending and/or analysis may be performed by a microprocessor that executes instructions stored in a computer-readable and/or machine-readable medium. At step 830, a user is provided with feedback on any trends in the recorded values. For example, in certain embodiments, a user is provided with audible, tactile, and/or visual feedback comprising at least one of a time of remaining safety in the region, a warning to exit the region, and indication of continued safety in the region, and an indication of impending danger.

As used above, the term “machine-readable medium” refers to any medium containing code or instructions that can be read or executed by a processor. Such a medium may take many forms, including, but not limited to, non-volatile media (e.g., magnetic disks or optical disks), volatile media (e.g., dynamic memory such as Random Access Memory), wired media (e.g., coaxial cables, copper wire, including the wires that comprise a bus, and fiber optics), wireless media (e.g., radio frequency, and other media in the electro-magnetic spectrum) and other forms of machine-readable media. Example of machine-readable media include a floppy disk, a hard disk, magnetic tape, a CD-ROM, DVD, and computer-readable media in general.

Those of skill in the art would appreciate that the various illustrative blocks, modules, elements, components, methods, and algorithms described herein may be implemented as electronic hardware, computer software, or combinations of both. Furthermore, these may be partitioned differently than what is described. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application.

It is understood that the specific order or hierarchy of steps or blocks in the processes disclosed is an illustration of exemplary approaches. Based upon design preferences, it is understood that the specific order or hierarchy of steps or blocks in the processes may be rearranged. The accompanying method claims present elements of the various steps in a sample order, and are not meant to be limited to the specific order or hierarchy presented. Some of the steps may be performed simultaneously.

Although the description above contains many specificities, these should not be construed as limiting the scope of the inventions but as merely providing illustrations of some of the presently preferred embodiments. Therefore, it will be appreciated that the scope of the present inventions fully encompasses other embodiments which may become obvious to those skilled in the art, and that the scope of the present inventions are accordingly to be limited by nothing other than the appended claims, in which reference to an element in the singular is not intended to mean “one and only one” unless explicitly so stated, but rather “one or more.” It is not necessary for a device or method to address each and every problem sought to be solved by the inventions, for it to be encompassed by the present claims. Furthermore, no element, component, or method step in the present disclosure is intended to be dedicated to the public regardless of whether the element, component, or method step is explicitly recited in the claims.

The previous description is provided to enable persons of ordinary skill in the art to practice the various aspects described herein. Various modifications to these aspects will be readily apparent to those skilled in the art, and the general principles defined herein may be applied to other aspects. Thus, the claims are not intended to be limited to the aspects shown herein, but are to be accorded the full scope consistent with the claim language. Headings and subheadings, if any, are used for convenience only and do not limit the inventions. All structural and functional equivalents to the elements of the various aspects described throughout the disclosure that are known or later come to be known to those of ordinary skill in the art are intended to be encompassed by the inventions.

What is claimed is:

1. A method, of monitoring emergency personnel, comprising:
acquiring values of levels of carboxyhemoglobin in a person's blood during a sampling period, the person being in a region near or at a source of carbon monoxide;
recording the values on machine-readable media;
determining a regression function for the values with respect to time;
determining at least one of an attribute of the function and a future value of a carboxyhemoglobin level in the person's blood, the future value being determined by extrapolation of the function; and
providing a user with safety information based on the at least one of the attribute of the function and the future value;
wherein the safety information comprises at least one of visible, tactile, and audible information; and
wherein the safety information comprises at least one of a time of remaining safety in the region, a warning to exit the region, an indication of continuing safety in the region, and an indication of impending danger.

2. The method of claim 1, wherein the attribute of the function comprises at least one of a derivative and an integral.

3. A machine-readable medium having machine-executable instructions for performing the method of claim 1.

4. A method, of monitoring emergency personnel, comprising:
during a sampling period and of a person in a region near or at a source of carbon monoxide, acquiring (a) values of levels of carboxyhemoglobin in the person's blood, and (b) a value of a parameter selected from the group consisting of the person's heart rate, an indicator of the person's peripheral perfusion, and ambient carbon monoxide;
recording the values of (a) and (b) on machine-readable media;
determining a regression function for at least one of: (i) each of (a) and (b) with respect to time; and (ii) a combination of (a) and (b) with respect to time;
determining at least one of: (i) an attribute of the function; and (ii) a future value of at least one of (a) and (b), the future value being determined by extrapolation of the function; and
providing a user with safety information based on the at least one of the attribute of the function and the future value;
wherein the safety information comprises at least one of visible, tactile, and audible information; and
wherein the safety information comprises at least one of a time of remaining safety in the region, a warning to exit the region, an indication of continuing safety in the region, and an indication of impending danger.

5. The method of claim 4, wherein the attribute of the function comprises at least one of a derivative and an integral.

6. The method of claim 4, further comprising:
determining an expected level of carboxyhemoglobin present in the person based on the value of ambient carbon monoxide.

7. A machine-readable medium having machine-executable instructions for performing the method of claim 4.

8. A device for monitoring emergency personnel comprising:
means for acquiring values of levels of carboxyhemoglobin in a person's blood during a sampling period, the person being in a region near or at a source of carbon monoxide;
means for recording the values on machine-readable media;
means for determining a regression function for the values with respect to time;
means for determining at least one of an attribute of the function and a future value of a carboxyhemoglobin level in the person's blood, the future value being determined by extrapolation of the function; and
means for providing a user with safety information based on the at least one of the attribute of the function and the future value;
wherein the safety information comprises at least one of visible, tactile, and audible information; and
wherein the safety information comprises at least one of a time of remaining safety in the region, a warning to exit the region, an indication of continuing safety in the region, and an indication of impending danger.

9. The device of claim 8, wherein the attribute of the function comprises at least one of a derivative and an integral.

10. A machine-readable medium having machine-executable instructions for performing steps corresponding to each of the means of claim 8.

11. A device for monitoring emergency personnel comprising:
means for acquiring, during a sampling period and of a person in a region near or at a source of carbon monoxide, (a) values of levels of carboxyhemoglobin in the person's blood, and (b) a value of a parameter selected from the group consisting of the person's heart rate, an indicator of the person's peripheral perfusion, and ambient carbon monoxide;
means for recording the values of (a) and (b) on machine-readable media;
means for determining a regression function for at least one of: (i) each of (a) and (b) with respect to time; and (ii) a combination of (a) and (b) with respect to time;
means for determining at least one of: (i) an attribute of the function; and (ii) a future value of at least one of (a) and (b), the future value being determined by extrapolation of the function; and
means for providing a user with safety information based on the at least one of the attribute of the function and the future value;
wherein the safety information comprises at least one of visible, tactile, and audible information; and
wherein the safety information comprises at least one of a time of remaining safety in the region, a warning to exit the region, an indication of continuing safety in the region, and an indication of impending danger.

12. The device of claim 11, wherein the attribute of the function comprises at least one of a derivative and an integral.

13. The device of claim 11, further comprising:
means for determining an expected level of carboxyhemoglobin present in the person based on the value of ambient carbon monoxide.

14. A machine-readable medium having machine-executable instructions for performing steps corresponding to each of the means of claim 11.

15. A device for monitoring emergency personnel, the device comprising:
a sensor configured to acquire values indicative of levels of carboxyhemoglobin in a person's blood during a sampling period, the person being in a region near or at a source of carbon monoxide;
a processor configured to determine a regression function for the values with respect to time, and to determine at least one of an attribute of the function and a future value of a carboxyhemoglobin level in the person's blood, the future value being determined by extrapolation of the function; and
an output device configured to provide a user with safety information based on at least one of the attribute of the function and the future value;
wherein the safety information comprises at least one of visible, tactile, and audible information; and
wherein the safety information comprises at least one of a time of remaining safety in the region, a warning to exit the region, an indication of continuing safety in the region, and an indication of impending danger.

16. The device of claim 15, wherein the attribute of the function comprises at least one of a derivative and an integral.

17. The device of claim 15, further comprising:
a receiver coupled with the processor; and
a transmitter,
wherein the transmitter transmits the acquired values to the receiver, and the receiver conveys the acquired values to the processor as inputs for at least the regression function.

18. The device of claim 15, further comprising:
a receiver coupled with the processor; and
a transmitter,
wherein the transmitter transmits at least one of the attribute of the function and the future value to the receiver, and the receiver conveys the at least one of the attribute of the function and the future value to the processor as inputs for the safety information.

19. The device of claim 15, further comprising:
a receiver coupled with the output device; and
a transmitter that transmits the safety information to the receiver;
wherein the receiver conveys the safety information to the output device.

20. A device for monitoring emergency personnel comprising:
a sensor configured to acquire during a sampling period and of a person in a region near or at a source of carbon monoxide, (a) values of levels of carboxyhemoglobin in the person's blood, and (b) a value of a parameter selected from the group consisting of the person's heart rate, an indicator of the person's peripheral perfusion, and ambient carbon monoxide;
a processor configured to determine a regression function for at least one of: (i) each of (a) and (b) with respect to time; and (ii) a combination of (a) and (b) with respect to time;
the processor is also configured to determine least one of: (i) an attribute of the function; and (ii) a future value of at least one of (a) and (b), the future value being determined by extrapolation of the function; and
an output device configured to provide a user with safety information based on the at least one of the attribute of the function and the future value;
wherein the safety information comprises at least one of visible, tactile, and audible information; and
wherein the safety information comprises at least one of a time of remaining safety in the region, a warning to exit the region, an indication of continuing safety in the region, and an indication of impending danger.

21. The device of claim 20, wherein the attribute of the function comprises at least one of a derivative and an integral.

22. The device of claim 20, further wherein the processor is further configured to determine expected levels of carboxyhemoglobin present in the person based on the value of ambient carbon monoxide.

23. The device of claim 20, further comprising:
a receiver coupled with the processor; and
a transmitter that transmits the acquired values to the receiver;
wherein the receiver conveys the acquired values to the processor as inputs for at least the regression function.

24. The device of claim 20, further comprising:
a receiver coupled with the processor; and
a transmitter that transmits at least one of the attribute of the function and the future value to the receiver, and the receiver conveys the at least one of the attribute of the function and the future value to the processor as inputs for the safety information.

25. The device of claim 20, further comprising:
a receiver coupled with the output device; and
a transmitter that transmits the safety information to the receiver;
wherein the receiver conveys the safety information to the output device.
